(19) United States
(12) Patent Application Publication
(10) Pub. No.: US 2011/0039304 A1
(43) Pub. Date: Feb. 17, 2011

(54) Methods to Generate Oligonucleotide Pools and Enrich Target Nucleic Acid Sequences

(75) Inventors: George M. Church, Brookline, MA (US); Duhee Bang, Seoul (KR); Jin Li, Palo Alto, CA (US)

Correspondence Address: BANNER & WITCOFF, LTD., 28 STATE STREET, SUITE 1800, BOSTON, MA, 02109-1701, US

(73) Assignee: President and Fellows of Harvard College, Cambridge, MA (US)

(21) Appl. No.: 12/855,137

(22) Filed: Aug. 12, 2010

Related U.S. Application Data
(60) Provisional application No. 61/233,350, filed on Aug. 12, 2009.

(51) Int. Cl. C12P 19/34

(52) U.S. Cl. 435/91.2

(57) ABSTRACT

Methods and compositions for generating oligonucleotide pools are provided. Methods and compositions for enriching target nucleic acid sequences are also provided.

### PRIORITY

This application claims priority from U.S. provisional patent application No. 61/233,350, filed Aug. 12, 2009, which is hereby incorporated herein by reference in its entirety for all purposes.

### STATEMENT OF GOVERNMENT INTERESTS

This invention was made with government support under HG003170 awarded by the National Institutes of Health. The government has certain rights in the invention.

### BACKGROUND

1. Field of the Invention

Embodiments of the present invention relate in general to methods and compositions for enriching nucleic acid sequences.

2. Description of Related Art

Current methods that exist to enrich nucleic acid sequences suffer from many drawbacks. For example, multiplex PCR has a low throughput which is very limited because it can enrich only up to tens of loci in one reaction. Padlock capture approaches in solution have been used to capture DNA (Porreca et al. (2007) Nat. Methods 11:931), but the processing procedure necessary to obtain the large number of oligonucleotides necessary for the preparation of padlock probes as well as capturing protocols are laborious. The initial expense of long oligonucleotide synthesis is also a limiting factor. Hybridization of target DNA fragments to the surface of a microarray has been also used to enrich target DNA. However, this approach is not cost effective as microarrays cannot be reused many times, and more than 20 μg of genomic DNA is needed for efficient hybridization.

### SUMMARY

Accordingly, the present invention provides methods to enrich one or more nucleic acid sequences (e.g., DNA and/or RNA sequences) from large stretches of nucleic acid sequences (e.g., DNA and/or RNA sequences (e.g., genes, genomes and the like)). The present invention further provides methods for generating pool(s) of oligonucleotide probes that are useful for synthesizing one or more gene(s) and/or genome(s).

In certain exemplary embodiments, methods for enriching a target nucleic acid sequence are provided. The methods include the steps of providing a double stranded oligonucleotide probe having a primer sequence at each 5′ end, wherein one strand includes a region that is complementary to the target nucleic acid sequence and includes a primer sequence having retrievable label and a phosphorothioate cap at its 5′ end, and wherein the primer sequence at the 5′ end of the other strand is phosphorylated, amplifying the probe, cleaving the strand having the 5′ phosphorylation to generate a single stranded probe having a 5′ retrievable label, hybridizing the single stranded probe having a 5′ retrievable label to a target nucleic acid sequence of interest, enriching the target nucleic acid sequence of interest by binding the 5′ retrievable label of the hybridized, single stranded probe having a 5′ retrievable label to a substrate, and releasing the nucleic acid sequence of interest from the substrate by denaturing. In certain aspects, the step of amplifying is performed by PCR. In other aspects, the retrievable label is biotin. In other aspects, step of enriching is performed by binding the label to streptavidin attached to a substrate. In yet other aspects, the substrate is a magnetic bead. In still other aspects, λ exonuclease is used to cleave the strand having the 5′ phosphorylation. In certain aspects, the target nucleic acid sequence is genomic DNA and/or genomic RNA.

In certain exemplary embodiments, methods for enriching a target nucleic acid sequence are provided. The methods include the steps of providing a circularized probe having a central region that is complementary to the target nucleic acid sequence; amplifying the probe by rolling circle amplification in the presence of a retrievable label to generate single stranded, amplified probe having a retrievable label attached thereto, hybridizing the single stranded, amplified probe having a retrievable label attached thereto to a target nucleic acid sequence of interest, enriching the target nucleic acid sequence of interest by binding the retrievable label to a substrate, and releasing the nucleic acid sequence of interest from the substrate by denaturing. In certain aspects, the retrievable label is added to the probe using a labeled primer or a labeled dNTP. In other aspects, the retrievable label is biotin. In still other aspects, the step of enriching is performed by binding the biotin label to streptavidin bound to a substrate. In yet other aspects, the substrate is a magnetic bead. In other aspects, the target nucleic acid sequence is genomic DNA and/or genomic RNA.

In certain exemplary embodiments, methods for enriching a target nucleic acid sequence are provided. The methods include the steps of providing a double stranded oligonucleotide probe having a primer sequence at each 5′ end, wherein one strand includes a region that is complementary to the target nucleic acid sequence and includes a primer sequence having retrievable label, and wherein the primer sequence at the 5′ end of the other strand is phosphorylated, amplifying the probe using an asymmetric ratio of forward primer to reverse primer (or an asymmetric ratio of reverse primer to forward primer) to generate a single stranded probe having a 5′ retrievable label, hybridizing the single stranded probe having a 5′ retrievable label to a target nucleic acid sequence of interest, enriching the target nucleic acid sequence of interest by binding the 5′ retrievable label of the hybridized, single stranded probe having a 5′ retrievable label to a substrate, and releasing the nucleic acid sequence of interest from the substrate by denaturing are provided. In certain aspects, the step of amplifying is performed by PCR. In other aspects, the single stranded probe is single stranded DNA. In yet other aspects, the ratio of forward primer to reverse primer is between about 100:1 and about 2:1, between about 20:1 and about 2:1, about 100:1, about 90:1, about 80:1, about 70:1, about 60:1, about 50:1, about 40:1, about 30:1, about 20:1, about 10:1 or about 5:1. In still other aspects, the ratio of reverse primer to forward primer is between about 100:1 and about 2:1, between about 20:1 and about 2:1, about 100:1, about 90:1, about 80:1, about 70:1, about 60:1, about 50:1, about 40:1, about 30:1, about 20:1, about 10:1 or about 5:1. In other aspects, the target nucleic acid sequence is genomic DNA and/or genomic RNA.

Further features and advantages of certain embodiments of the present invention will become more fully apparent in the following description of the embodiments and drawings thereof, and from the claims.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIGS. 1A-1C schematically depict generation of single stranded (ss) DNA from a DNA chip by copying. A) Oligonucleotide sequences (e.g., 70-mers) are synthesized on a microarray. Target sequences (e.g., exonic sequences, middle 40-mer) are flanked on either side by common sequences (15-mers). B) Common primers are annealed. C) The second strand of oligonucleotide sequences are synthesized by extending the annealed primers. Extended single stranded oligonucleotide sequences are denatured and released to a tube.

FIGS. 2A-2E schematically depict a biotin-coupled capturing strategy. A) The released ssDNA is used for PCR amplification. B) For PCR amplification, one PCR primer is biotinylated, and the other is phosphorylated. C) The PCR product is digested with lambda-exonuclease and the biotinylated single strand will be used in hybridization. D) The biotinylated ssDNA is hybridized with genomic DNA that is randomly sheared, end repaired, and ligated with adapters. After hybridization, streptavidin coated magnetic beads are added to enrich selected genomic DNA. E) The hybridized genomic DNA is released from the biotinylated DNA-bound beads by denaturing, and then amplified and subjected to sequencing.

FIGS. 3A-3G schematically depict a rolling circle amplification (RCA) generated ssDNA assisted capturing strategy. A) the collected ssDNA is circularized using circ-ligase. B) The circularized ssDNA is subjected to the RCA reaction. During the RCA reaction, biotinylated primer and/or biotinylated dNTP (available from NEB, Invitrogen) to provide a retrievable label that allows purification of captured DNA sequences. C) The biotinylated ssDNA is hybridized with target DNA (e.g., genomic DNA can be randomly sheared, end repaired, and ligated with adapters). After hybridization, streptavidin coated magnetic beads are added to enrich selected genomic DNA. D) The hybridized genomic DNA is released from the biotinylated bound beads by denaturing are amplified and subjected to sequencing. E) The circularized, ssDNA is subjected to the RCA reaction. During the RCA reaction (without adding biotinylated primer or biotinylated dNTP) RCA products can be saturated up to the point of precipitation by either performing hyperbranched RCA or long period linear RCA. F) The precipitated RCA products can be used to enrich one or more target DNA regions. Centrifugation at high speeds can be used to separate unbound DNA and target DNA bound with the RCA-processed probes. The RCA produced ssDNA is hybridized with genomic DNA that is randomly sheared, end repaired and ligated with adapters. G) the hybridized DNA is amplified using adapter sequences and subjected to sequencing.

### DETAILED DESCRIPTION

The methods and compositions described herein provide the advantages of: (1) dramatically reducing cost, as the DNA probes have a low initial financial investment and are amplifiable; (2) utilizing sub-μg amounts of human genomic DNA, as the reactions can be performed in aqueous phase, very compactly using a single tube; (3) having very uniform capturing efficiency from one region to another as demonstrated by microarray surface hybridization methods; and (4) being readily scalable and useful with next generation sequencing platforms.

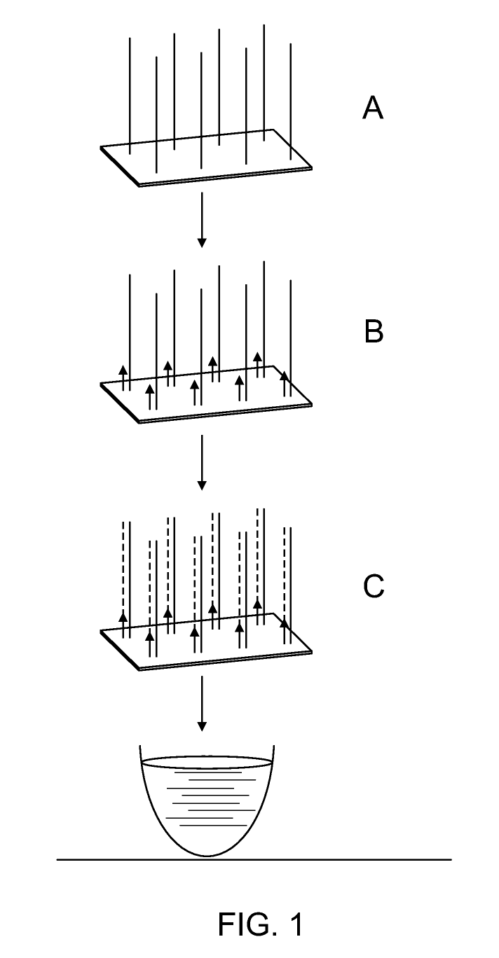

In certain exemplary embodiments, a method for generating single stranded DNA (ssDNA) from one or more DNA microarrays is provided. In certain aspects, ssDNA is generated from a microchip by producing DNA on a microarray, and removing ssDNA from the array by denaturing and elution. The reverse complementary copy of the oligonucleotides on the microarray can be made by this method (FIG. 1). In other aspects, ssDNA is generated using an asymmetric ratio of PCR primers (e.g., forward primer:reverse primer is about 10:1), and then by saturating the PCR product (i.e., after depleting the reverse primer, the remaining PCR steps will generate only ssDNA).

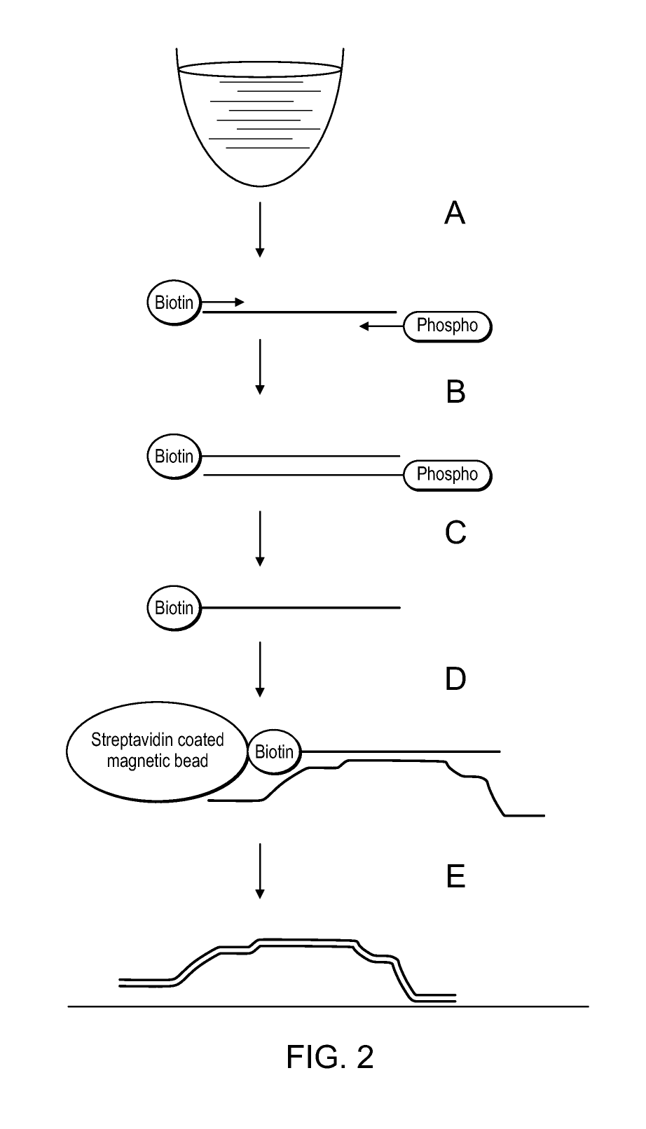

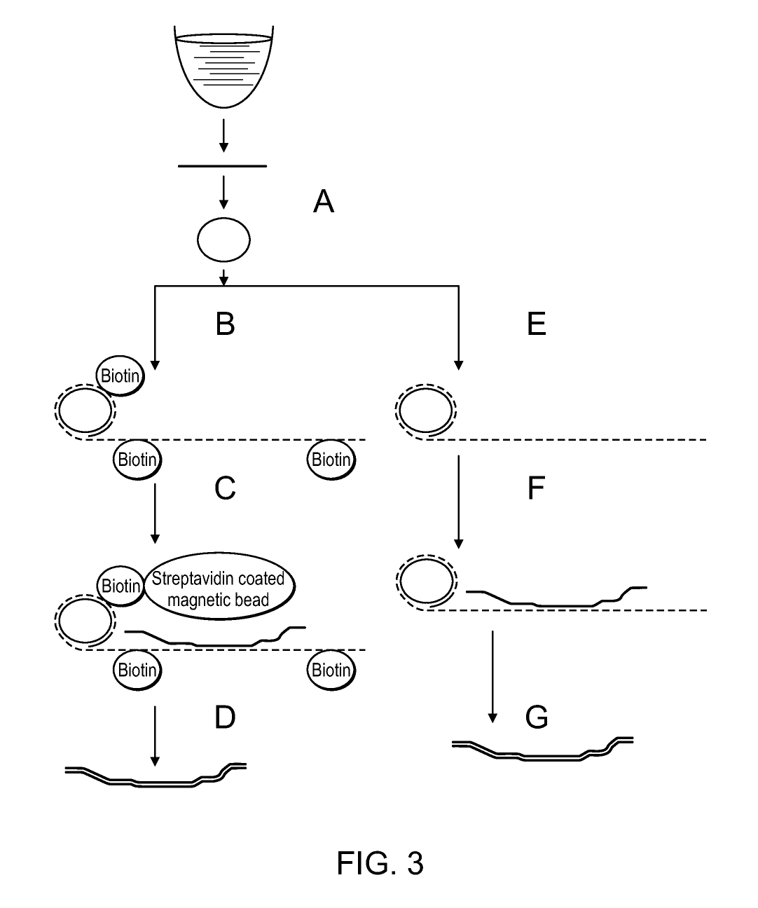

In certain exemplary embodiments, a method for enriching target DNA using ssDNA probes is provided. In certain aspects, a target nucleic acid sequence (e.g., DNA and/or RNA) is enriched using biotin-coupled ssDNA (FIG. 2). In other aspects, a target nucleic acid sequence (e.g., DNA and/or RNA) is enriched using ssDNA generated by rolling circle amplification (RCA) (FIG. 3). Target nucleic acid sequences include, but are not limited to, one or more genomes (e.g., genomic DNA and/or genomic RNA), genes, exons, introns, untranslated regions, intergenic regions, messenger RNAs (mRNAs), transfer RNAs, ribosomal RNAs, ribozymes, small interfering RNAs (siRNAs), cDNAs and the like.

Samples or specimens containing a target nucleic acid sequence, such as fragments of genomic DNA and/or genomic RNA, may come from a wide variety of sources for use with the present invention, including, but not limited to, cell cultures, animal or plant tissues, patient biopsies, environmental samples, and the like. Samples are prepared using conventional techniques, which typically depend on the source from which a sample or specimen is taken.

Prior to carrying out reactions on a sample, it will often be desirable to perform one or more sample preparation operations upon the sample. In certain exemplary embodiments, sample preparation operations will include such manipulations as extraction of intracellular material, e.g., nucleic acids from a sample.

“Sample” means a quantity of material from a biological, environmental, medical, or patient source in which detection or measurement of target nucleic acids is sought. On the one hand it is meant to include a specimen or culture (e.g., microbiological cultures). On the other hand, it is meant to include both biological and environmental samples. A sample may include a specimen of synthetic origin. Biological samples may be viral, bacterial, fungal or animal, including human, fluid, solid (e.g., stool or tissue), as well as liquid and solid food and feed products and ingredients such as dairy items, vegetables, meat and meat by-products, and waste. Biological samples may include materials taken from a patient including, but not limited to cultures, cells, tissues, blood, saliva, cerebral spinal fluid, pleural fluid, milk, lymph, sputum, semen, needle aspirates, and the like. Biological samples may be obtained from all of the various families of domestic animals, as well as feral or wild animals, including, but not limited to, such animals as ungulates, bear, fish, rodents, etc. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

In certain aspects, it will be necessary to extract the nucleic acids from the sample prior to continuing with the various sample preparation operations. Accordingly, following sample collection, nucleic acids may be liberated from the sample into a crude extract, followed by additional treatments to prepare the sample for subsequent operations, e.g., denaturation of contaminating (DNA binding) proteins, purification, filtration, desalting, and the like. Liberation of nucleic acids from the sample cells or viruses, and denaturation of DNA binding proteins may generally be performed by chemical, physical, or electrolytic lysis methods. For example, chemical methods generally employ lysing agents to disrupt the cells and extract the nucleic acids from the cells, followed by treatment of the extract with chaotropic salts such as guanidinium isothiocyanate or urea to denature any contaminating and potentially interfering proteins. Generally, where chemical extraction and/or denaturation methods are used, the appropriate reagents may be incorporated within a sample preparation chamber, a separate accessible chamber, or may be externally introduced.

Following extraction, it will often be desirable to separate the nucleic acids from other elements of the crude extract, e.g., denatured proteins, cell membrane particles, salts, and the like. Removal of particulate matter is generally accomplished by filtration, flocculation or the like. A variety of filter types may be readily incorporated into the device. Further, where chemical denaturing methods are used, it may be desirable to desalt the sample prior to proceeding to the next step. Desalting of the sample, and isolation of the nucleic acid may generally be carried out in a single step, e.g., by binding the nucleic acids to a solid phase and washing away the contaminating salts or performing gel filtration chromatography on the sample, passing salts through dialysis membranes, and the like. Suitable solid supports for nucleic acid binding include, e.g., diatomaceous earth, silica (i.e., glass wool), or the like. Suitable gel exclusion media, also well known in the art and may also be readily incorporated into the methods and compositions described herein (commercially available from, e.g., Pharmacia and Sigma Chemical).

Methods for enriching target nucleic acid sequences include the use of molecular inversion probe (MIP) technology. “MIP technology” refers to a high-throughput genotyping technology capable of interrogating single base changes of a nucleic acid sequence of interest (e.g., single nucleotide polymorphisms, mutations and the like) on a large scale. Methods of using molecular inversion probe technology in highly multiplexed genotyping of SNPs are known. See Hardenbol et al. Genome Res. (2005) 15:269 and Hardenbol et al. (2003) Nat. Biotechnol. 21:673. The use of molecular inversion probe technology in allele quantification is also known. See Wang et al. (2005) Nucl. Acids Res. 33(21).

Generally, MIP technology is directed to the use of a single oligonucleotide probe with recognition sequences at each terminus and optionally, one or more barcode sequences. The probe is hybridized with a target (e.g., genomic) nucleic acid sequence such that it forms a circular structure, with the ends of the probe abutting. This leaves a single base gap at the location of a nucleic acid sequence of interest. Probe designs having a gap length greater than one base are also useful in assays in which it is desirable to capture longer target DNA sequences. This gapped-duplex is then tested in four separate reactions, each with a single dNTP species present, in which successful polymerization and ligation provides allelic differentiation. The probes are subsequently released from the target nucleic acid sequence and those that have been covalently circularized in the correct allele/nucleotide reaction combination are amplified using a “universal” PCR primer pair. Tags are selected to have a similar Tₘ and base composition and to be maximally orthogonal in sequence complementarity.

According to one exemplary embodiment, molecular inversion probes are used based on the methods described in Hardenbol, Nature Biotech., Vol. 21, No. 6., 6 Jun. 1993, Hardenbol et al., Genome Research, 2005; 15(2):269-75; Fakhrai et al. (2003) Nature Biotech. 21(6):673 and Wang et al. (2005) Nucl. Acids Res. 33:e183. For SNP detection, a single probe is used to detect both alleles of each SNP. The probe includes two regions of homology to a target nucleic acid sequence (e.g., a SNP, an antibiotic resistance gene or the like) located at the termini or end of the probe and two PCR primer regions common to all probes, and two common cleavage sites. A universal detection tag sequence can optionally be included for array detection of amplified probe. Cleavage sites are used to release the circularized probe from a target nucleic acid sequence and for post-amplification processing.

According to exemplary embodiment, methods are provided whereby shareable probe pools are used. According to this aspect, large quantities and diverse numbers of MIP probes are generated on oligonucleotide chips (e.g. Agilent) in a way that is poolable & amplifiable (and hence easily shared). Each MIP oligo is flanked by universal oligos for amplification which can be removed. The following approaches are used to isolate the appropriate strand of the double stranded PCR products as well as to remove the universal primer regions mentioned above. (1) using one or more 3′ phosphothioate nucleotides on one of the two primers, (2) using exonucleases sensitive to 3′ or 5′ overhang (or lack thereof). One primer has one or more dU and can be removed by USER (which is a mixture of uracil DNA glycosylase and DNA glycosylase-lyase Endonuclease VIII) then the other primer has rU which can be cleaved by alkali. (3) using solid phase immobilization (e.g. magnetic bead streptavidin) of one primer with selective release of the other strand using alkali or heat to melt the base-pairs. (4) using asymmetric PCR (using an excess of the desired strand's primer) and (5) using separation by size and/or electrophoretic differences of the two strands by engineering the oligos to have different lengths (either by use of the rU or dU methods or 2′O methyl groups to block PCR extension beyond the 2′Ome.

According to certain aspects of the present invention, molecular inversion probes can be manufactured having gaps larger than one nucleotide and without extending the length of the molecular inversion probe. According to one aspect, the single stranded regions of the MIP during ligation reaction are free to extend far beyond the usual 0.34 nm/base and are free to rotate, unlike perfect CCC. Alternatively, very small DNA circles can be made according to the methods described in Bates et al. (1989) EMBO J. 8:1861. According to the present invention, smaller MIP probes aimed at large targets are believed to perform better in the range of 300 to 900 base pairs, which is advantageous for exons and other conserved elements.

According to the present invention, a mixture of sample nucleic acid sequences, a plurality of probes and thermostable ligase and polymerase is heat denatured and brought to annealing temperature. Two sequences targeting each terminus of the probe hybridize to complementary sites in the target nucleic acid sequence, creating a circular conformation with a single-nucleotide gap between the termini of the probe. According to an alternate embodiment, the gap may be greater than one nucleotide. The genomic DNA is then split into four separate samples. Unlabeled dATP, dCTP, dGTP or dTTP is added to each of four samples. In reactions where the added nucleotide is complementary to the single base gap, DNA polymerase adds the nucleotide and DNA ligase closes the gap to form a covalently closed circular molecule that encircles the genomic strand to which it is hybridized. Exonucleases are added to digest linear probes in reactions where the added nucleotide was not complementary to the gap and excess linear probe in reactions where circular molecules were formed. The reactions are then heated to inactivate the exonucleases. To release probes from sample nucleic acid sequences, uracil-N-glycosylase is added to depurinate the uracil residues in the probes. The mixture is then heated to cleave the molecule at the abasic site and release it from sample nucleic acid sequences. Alternatively, the molecule can be removed from the sample nucleic acid sequences through methods other than cleavage, thereby leaving the molecule in its circular form.

In certain exemplary embodiments, methods for amplifying nucleic acid sequences are provided. Exemplary methods for amplifying nucleic acids include the polymerase chain reaction (PCR) (see, e.g., Mullis et al. (1986) Cold Spring Harb. Symp. Quant. Biol. 51 Pt 1:263 and Cleary et al. (2004) Nature Methods 1:241; and U.S. Pat. Nos. 4,683,195 and 4,683,202), anchor PCR, RACE PCR, ligation chain reaction (LCR) (see, e.g., Landegran et al. (1988) Science 241:1077-1080; and Nakazawa et al. (1994) Proc. Natl. Acad. Sci. U.S.A. 91:360-364), self sustained sequence replication (Guatelli et al. (1990) Proc. Natl. Acad. Sci. U.S.A. 87:1874), transcriptional amplification system (Kwoh et al. (1989) Proc. Natl. Acad. Sci. U.S.A. 86:1173), Q-Beta Replicase (Lizardi et al. (1988) BioTechnology 6:1197), recursive PCR (Jaffe et al. (2000) J. Biol. Chem. 275:2619; and Williams et al. (2002) J. Biol. Chem. 277:7790), the amplification methods described in U.S. Pat. Nos. 6,391,544, 6,365,375, 6,294,323, 6,261,797, 6,124,090 and 5,612,199, isothermal amplification (e.g., rolling circle amplification (RCA), hyperbranched rolling circle amplification (HRCA), strand displacement amplification (SDA), helicase-dependent amplification (HDA), PWGA) or any other nucleic acid amplification method using techniques well known to those of skill in the art.

Several suitable RCA methods are known in the art. For example, linear RCA amplifies circular DNA by polymerase extension of a complementary primer. This process generates concatemerized copies of the circular DNA template such that multiple copies of a DNA sequence arranged end to end in tandem are generated. Exponential RCA is similar to the linear process except that it uses a second primer of identical sequence to the DNA circle (Lizardi et al. (1998) Nat. Genet. 19:225). This two-primer system achieves isothermal, exponential amplification. Exponential RCA has been applied to the amplification of non-circular DNA through the use of a linear probe that binds at both of its ends to contiguous regions of a target DNA followed by circularization using DNA ligase (i.e., padlock RCA) (Nilsson et al. (1994) Science 265(5181):2085). Hyperbranched RCA uses a second primer complementary to the rolling circle replication (RCR) product. This allows RCR products to be replicated by a strand-displacement mechanism, which can yield a billion-fold amplification in an isothermal reaction (Dahl et al. (2004) Proc. Natl. Acad. Sci. U.S.A. 101(13):4548).

“Polymerase chain reaction,” or “PCR,” refers to a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g., exemplified by the references: McPherson et al., editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature greater than 90° C., primers annealed at a temperature in the range 50-75° C., and primers extended at a temperature in the range 72-78° C.

The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g., 200 mL, to a few hundred microliters, e.g., 200 microliters. “Reverse transcription PCR,” or “RT-PCR,” means a PCR that is preceded by a reverse transcription reaction that converts a target RNA to a complementary single stranded DNA, which is then amplified, e.g., Tecott et al., U.S. Pat. No. 5,168,038. “Real-time PCR” means a PCR for which the amount of reaction product, i.e., amplicon, is monitored as the reaction proceeds. There are many forms of real-time PCR that differ mainly in the detection chemistries used for monitoring the reaction product, e.g., Gelfand et al., U.S. Pat. No. 5,210,015 (“Taqman”); Wittwer et al., U.S. Pat. Nos. 6,174,670 and 6,569,627 (intercalating dyes); Tyagi et al., U.S. Pat. No. 5,925,517 (molecular beacons). Detection chemistries for real-time PCR are reviewed in Mackay et al., Nucleic Acids Research, 30:1292-1305 (2002). “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al. (1999) Anal. Biochem., 273:221-228 (two-color real-time PCR). Usually, distinct sets of primers are employed for each sequence being amplified. “Quantitative PCR” means a PCR designed to measure the abundance of one or more specific target sequences in a sample or specimen. Techniques for quantitative PCR are well-known to those of ordinary skill in the art, as exemplified in the following references: Freeman et al., Biotechniques, 26:112-126 (1999); Becker-Andre et al., Nucleic Acids Research, 17:9437-9447 (1989); Zimmerman et al., Biotechniques, 21:268-279 (1996); Diviacco et al., Gene, 122:3013-3020 (1992); Becker-Andre et al., Nucleic Acids Research, 17:9437-9446 (1989); and the like.

In certain embodiments, methods of determining the nucleic acid sequence of one or more nucleic acid sequences (e.g., target nucleic acid sequences) are provided. Determination of a target nucleic acid sequence can be performed using variety of sequencing methods known in the art including, but not limited to, sequencing by hybridization (SBH), sequencing by ligation (SBL), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (PCT/US05/27695), multiplex sequencing (U.S. Ser. No. 12/027,039, filed Feb. 6, 2008; Porreca et al (2007) Nat. Methods 4:931), polymerized colony (POLONY) sequencing (U.S. Pat. Nos. 6,432,360, 6,485,944 and 6,511,803, and PCT/US05/06425); nanogrid rolling circle sequencing (ROLONY) (U.S. Ser. No. 12/120,541, filed May 14, 2008), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout) and the like. High-throughput sequencing methods, e.g., on cyclic array sequencing using platforms such as Roche 454, Illumina Solexa, AB-SOLiD, Helicos, Polonator platforms and the like, can also be utilized. High-throughput sequencing methods are described in U.S. Ser. No. 61/162,913, filed Mar. 24, 2009. A variety of light-based sequencing technologies are known in the art (Landegren et al. (1998) Genome Res. 8:769-76; Kwok (2000) Pharmocogenomics 1:95-100; and Shi (2001) Clin. Chem. 47:164-172).

Terms and symbols of nucleic acid chemistry, biochemistry, genetics, and molecular biology used herein follow those of standard treatises and texts in the field, e.g., Komberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See Kanehisa (1984) Nucl. Acids Res. 12:203. According to the present invention, useful MIP primer sequences hybridize to sequences that flank the nucleotide base or series of bases to be captured.

“Complex” means an assemblage or aggregate of molecules in direct or indirect contact with one another. In one aspect, “contact,” or more particularly, “direct contact,” in reference to a complex of molecules or in reference to specificity or specific binding, means two or more molecules are close enough so that attractive noncovalent interactions, such as van der Waal forces, hydrogen bonding, ionic and hydrophobic interactions, and the like, dominate the interaction of the molecules. In such an aspect, a complex of molecules is stable in that under assay conditions the complex is thermodynamically more favorable than a non-aggregated, or non-complexed, state of its component molecules. As used herein, “complex” refers to a duplex or triplex of polynucleotides or a stable aggregate of two or more proteins. In regard to the latter, a complex is formed by an antibody specifically binding to its corresponding antigen.

“Duplex” means at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. In one aspect, stable duplex means that a duplex structure is not destroyed by a stringent wash, e.g., conditions including temperature of about 5° C. less that the Tₘ of a strand of the duplex and low monovalent salt concentration, e.g., less than 0.2 M, or less than 0.1 M. “Perfectly matched” in reference to a duplex means that the polynucleotide or oligonucleotide strands making up the duplex form a double stranded structure with one another such that every nucleotide in each strand undergoes Watson-Crick base pairing with a nucleotide in the other strand. The term “duplex” comprehends the pairing of nucleoside analogs, such as deoxyinosine, nucleosides with 2-aminopurine bases, PNAs, and the like, that may be employed. A “mismatch” in a duplex between two oligonucleotides or polynucleotides means that a pair of nucleotides in the duplex fails to undergo Watson-Crick bonding.

“Genetic locus,” or “locus” in reference to a genome or target polynucleotide, means a contiguous subregion or segment of the genome or target polynucleotide. As used herein, genetic locus, or locus, may refer to the position of a nucleotide, a gene, or a portion of a gene in a genome, including mitochondrial DNA, or it may refer to any contiguous portion of genomic sequence whether or not it is within, or associated with, a gene. In one aspect, a genetic locus refers to any portion of genomic sequence, including mitochondrial DNA, from a single nucleotide to a segment of few hundred nucleotides, e.g. 100-300, in length. Usually, a particular genetic locus may be identified by its nucleotide sequence, or the nucleotide sequence, or sequences, of one or both adjacent or flanking regions. In another aspect, a genetic locus refers to the expressed nucleic acid product of a gene, such as an RNA molecule or a cDNA copy thereof.

“Hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” “Hybridization conditions” will typically include salt concentrations of less than about 1 M, more usually less than about 500 mM and even more usually less than about 200 mM. Hybridization temperatures can be as low as 5° C., but are typically greater than 22° C., more typically greater than about 30° C., and often in excess of about 37° C. Hybridizations are usually performed under stringent conditions, i.e., conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tₘ for the specific sequence at s defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5XSSPE (750 mM NaCl, 50 mM Na phosphate, 5 mM EDTA, pH 7.4) and a temperature of 25-30° C. are suitable for allele-specific probe hybridizations. For stringent conditions, see for example, Sambrook, Fritsche and Maniatis, Molecular Cloning A Laboratory Manual, 2nd Ed. Cold Spring Harbor Press (1989) and Anderson Nucleic Acid Hybridization, 1^{st} Ed., BIOS Scientific Publishers Limited (1999). “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA.

“Hybridization-based assay” means any assay that relies on the formation of a stable complex as the result of a specific binding event. In one aspect, a hybridization-based assay means any assay that relies on the formation of a stable duplex or triplex between a probe and a target nucleotide sequence for detecting or measuring such a sequence. In one aspect, probes of such assays anneal to (or form duplexes with) regions of target sequences in the range of from 8 to 100 nucleotides; or in other aspects, they anneal to target sequences in the range of from 8 to 40 nucleotides, or more usually, in the range of from 8 to 20 nucleotides. A “probe” in reference to a hybridization-based assay means a polynucleotide that has a sequence that is capable of forming a stable hybrid (or triplex) with its complement in a target nucleic acid and that is capable of being detected, either directly or indirectly.

Hybridization-based assays include, without limitation, assays that use the specific base-pairing of one or more oligonucleotides as target recognition components, such as polymerase chain reactions, NASBA reactions, oligonucleotide ligation reactions, single-base extension reactions, circularizable probe reactions, allele-specific oligonucleotide hybridizations, either in solution phase or bound to solid phase supports, such as microarrays or microbeads, and the like. An important subset of hybridization-based assays include such assays that have at least one enzymatic processing step after a hybridization step. Hybridization-based assays of this subset include, without limitation, polymerase chain reactions, NASBA reactions, oligonucleotide ligation reactions, cleavase reactions, e.g., in INVADER™ assays, single-base extension reactions, probe circularization reactions, and the like. There is extensive guidance in the literature on hybridization-based assays, e.g., Hames et al., editors, Nucleic Acid Hybridization a Practical Approach (IRL Press, Oxford, 1985); Tijssen, Hybridization with Nucleic Acid Probes, Parts I & II (Elsevier Publishing Company, 1993); Hardiman, Microarray Methods and Applications (DNA Press, 2003); Schena, editor, DNA Microarrays a Practical Approach (IRL Press, Oxford, 1999); and the like. In one aspect, hybridization-based assays are solution phase assays; that is, both probes and target sequences hybridize under conditions that are substantially free of surface effects or influences on reaction rate. A solution phase assay includes circumstances where either probes or target sequences are attached to microbeads such that the attached sequences have substantially the same environment (e.g., permitting reagent access, etc.) as free sequences. In another aspect, hybridization-based assays include immunoassays wherein antibodies employ nucleic acid reporters based on amplification. In such assays, antibody probes specifically bind to target molecules, such as proteins, in separate reactions, after which the products of such reactions (i.e., antibody-protein complexes) are combined and nucleic acid reporters are amplified. Preferably, such nucleic acid reporters include oligonucleotide tags that are converted enzymatically into labeled oligonucleotide tags for analysis on a microarray, as described below. The following exemplary references disclose antibody-nucleic acid conjugates for immunoassays: Baez et al., U.S. Pat. No. 6,511,809; Sano et al., U.S. Pat. No. 5,665,539; Eberwine et al., U.S. Pat. No. 5,922,553; Landegren et al., U.S. Pat. No. 6,558,928; Landegren et al., U.S. Patent Pub. 2002/0064779; and the like. In particular, the two latter patent publications by Landegren et al. disclose steps of forming amplifiable probes after a specific binding event.

“Kit” refers to any delivery system for delivering materials or reagents for carrying out a method of the invention. In the context of assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials for assays of the invention. Such contents may be delivered to the intended recipient together or separately. For example, a first container may contain an enzyme for use in an assay, while a second container contains probes.

“Ligation” means to form a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon of a terminal nucleotide of one oligonucleotide with 3′ carbon of another oligonucleotide. A variety of template-driven ligation reactions are described in the following references: Whitely et al., U.S. Pat. No. 4,883,750; Letsinger et al., U.S. Pat. No. 5,476,930; Fung et al., U.S. Pat. No. 5,593,826; Kool, U.S. Pat. No. 5,426,180; Landegren et al., U.S. Pat. No. 5,871,921; Xu and Kool (1999) Nucl. Acids Res. 27:875; Higgins et al., Meth. in Enzymol. (1979) 68:50; Engler et al. (1982) The Enzymes, 15:3 (1982); and Namsaraev, U.S. Patent Pub. 2004/0110213.

“Microarray” refers in one embodiment to a type of multiplex assay product that comprises a solid phase support having a substantially planar surface on which there is an array of spatially defined non-overlapping regions or sites that each contain an immobilized hybridization probe. “Substantially planar” means that features or objects of interest, such as probe sites, on a surface may occupy a volume that extends above or below a surface and whose dimensions are small relative to the dimensions of the surface. For example, beads disposed on the face of a fiber optic bundle create a substantially planar surface of probe sites, or oligonucleotides disposed or synthesized on a porous planar substrate creates a substantially planar surface. Spatially defined sites may additionally be “addressable” in that its location and the identity of the immobilized probe at that location are known or determinable. Probes immobilized on microarrays include nucleic acids, such as oligonucleotide barcodes, that are generated in or from an assay reaction. Typically, the oligonucleotides or polynucleotides on microarrays are single stranded and are covalently attached to the solid phase support, usually by a 5′-end or a 3′-end. The density of non-overlapping regions containing nucleic acids in a microarray is typically greater than 100 per cm², and more preferably, greater than 1000 per cm². Microarray technology relating to nucleic acid probes is reviewed in the following exemplary references: Schena, Editor, Microarrays: A Practical Approach (IRL Press, Oxford, 2000); Southern, Current Opin. Chem. Biol., 2: 404-410 (1998); Nature Genetics Supplement, 21:1-60 (1999); and Fodor et al., U.S. Pat. Nos. 5,424,186; 5,445,934; and 5,744,305. A microarray may comprise arrays of microbeads, or other microparticles, alone or disposed on a planar surface or in wells or other physical configurations that can be sued to separate the beads. Such microarrays may be formed in a variety of ways, as disclosed in the following exemplary references: Brenner et al. (2000) Nat. Biotechnol. 18:630; Tulley et al., U.S. Pat. No. 6,133,043; Stuelpnagel et al., U.S. Pat. No. 6,396,995; Chee et al., U.S. Pat. No. 6,544,732; and the like. In one format, microarrays are formed by randomly disposing microbeads having attached oligonucleotides on a surface followed by determination of which microbead carries which oligonucleotide by a decoding procedure, e.g. as disclosed by Gunderson et al., U.S. Patent Pub. No. 2003/0096239.

“Microarrays” or “arrays” can also refer to a heterogeneous pool of nucleic acid molecules that is distributed over a support matrix. The nucleic acids can be covalently or noncovalently attached to the support. Preferably, the nucleic acid molecules are spaced at a distance from one another sufficient to permit the identification of discrete features of the array. Nucleic acids on the array may be non-overlapping or partially overlapping. Methods of transferring a nucleic acid pool to support media is described in U.S. Pat. No. 6,432,360. Bead based methods useful in the present invention are disclosed in PCT US05/04373.

“Amplifying” includes the production of copies of a nucleic acid molecule of the array or a nucleic acid molecule bound to a bead via repeated rounds of primed enzymatic synthesis. “In situ” amplification indicated that the amplification takes place with the template nucleic acid molecule positioned on a support or a bead, rather than in solution. In situ amplification methods are described in U.S. Pat. No. 6,432,360.

“Support” can refer to a matrix upon which nucleic acid molecules of a nucleic acid array are placed. The support can be solid or semi-solid or a gel. “Semi-solid” refers to a compressible matrix with both a solid and a liquid component, wherein the liquid occupies pores, spaces or other interstices between the solid matrix elements. Semi-solid supports can be selected from polyacrylamide, cellulose, polyamide (nylon) and crossed linked agarose, dextran and polyethylene glycol.

“Randomly-patterned” or “random” refers to non-ordered, non-Cartesian distribution (in other words, not arranged at pre-determined points along the x- or y-axes of a grid or at defined “clock positions,” degrees or radii from the center of a radial pattern) of nucleic acid molecules over a support, that is not achieved through an intentional design (or program by which such design may be achieved) or by placement of individual nucleic acid features. Such a “randomly-patterned” or “random” array of nucleic acids may be achieved by dropping, spraying, plating or spreading a solution, emulsion, aerosol, vapor or dry preparation comprising a pool of nucleic acid molecules onto a support and allowing the nucleic acid molecules to settle onto the support without intervention in any manner to direct them to specific sites thereon. Arrays of the invention can be randomly patterned or random.

“Heterogeneous” refers to a population or collection of nucleic acid molecules that comprises a plurality of different sequences. According to one aspect, a heterogeneous pool of nucleic acid molecules results from a preparation of RNA or DNA from a cell which may be unfractionated or partially-fractionated.

“Nucleoside” as used herein includes the natural nucleosides, including 2′-deoxy and 2′-hydroxyl forms, e.g. as described in Komberg and Baker, DNA Replication, 2nd Ed. (Freeman, San Francisco, 1992). “Analogs” in reference to nucleosides includes synthetic nucleosides having modified base moieties and/or modified sugar moieties, e.g., described by Scheit, Nucleotide Analogs (John Wiley, New York, 1980); Uhlman and Peyman, Chemical Reviews, 90:543-584 (1990), or the like, with the proviso that they are capable of specific hybridization. Such analogs include synthetic nucleosides designed to enhance binding properties, reduce complexity, increase specificity, and the like. Polynucleotides comprising analogs with enhanced hybridization or nuclease resistance properties are described in Uhlman and Peyman (cited above); Crooke et al., Exp. Opin. Ther. Patents, 6: 855-870 (1996); Mesmaeker et al., Current Opinion in Structural Biology, 5:343-355 (1995); and the like. Exemplary types of polynucleotides that are capable of enhancing duplex stability include oligonucleotide phosphoramidates (referred to herein as “amidates”), peptide nucleic acids (referred to herein as “PNAs”), oligo-2′-O-alkylribonucleotides, polynucleotides containing C-5 propynylpyrimidines, locked nucleic acids (LNAs), and like compounds. Such oligonucleotides are either available commercially or may be synthesized using methods described in the literature.

“Oligonucleotide” or “polynucleotide,” which are used synonymously, means a linear polymer of natural or modified nucleosidic monomers linked by phosphodiester bonds or analogs thereof. The term “oligonucleotide” usually refers to a shorter polymer, e.g., comprising from about 3 to about 100 monomers, and the term “polynucleotide” usually refers to longer polymers, e.g., comprising from about 100 monomers to many thousands of monomers, e.g., 10,000 monomers, or more. Oligonucleotides comprising probes or primers usually have lengths in the range of from 12 to 60 nucleotides, and more usually, from 18 to 40 nucleotides. Oligonucleotides and polynucleotides may be natural or synthetic. Oligonucleotides and polynucleotides include deoxyribonucleosides, ribonucleosides, and non-natural analogs thereof, such as anomeric forms thereof, peptide nucleic acids (PNAs), and the like, provided that they are capable of specifically binding to a target genome by way of a regular pattern of monomer-to-monomer interactions, such as Watson-Crick type of base pairing, base stacking, Hoogsteen or reverse Hoogsteen types of base pairing, or the like.

Usually nucleosidic monomers are linked by phosphodiester bonds. Whenever an oligonucleotide is represented by a sequence of letters, such as “ATGCCTG,” it will be understood that the nucleotides are in 5′ to 3′ order from left to right and that “A” denotes deoxyadenosine, “C” denotes deoxycytidine, “G” denotes deoxyguanosine, “T” denotes deoxythymidine, and “U” denotes the ribonucleoside, uridine, unless otherwise noted. Usually oligonucleotides comprise the four natural deoxynucleotides; however, they may also comprise ribonucleosides or non-natural nucleotide analogs. It is clear to those skilled in the art when oligonucleotides having natural or non-natural nucleotides may be employed in methods and processes described herein. For example, where processing by an enzyme is called for, usually oligonucleotides consisting solely of natural nucleotides are required. Likewise, where an enzyme has specific oligonucleotide or polynucleotide substrate requirements for activity, e.g., single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al., Molecular Cloning, Second Edition (Cold Spring Harbor Laboratory, New York, 1989), and like references. Oligonucleotides and polynucleotides may be single stranded or double stranded.

“Oligonucleotide tag” or “tag” means an oligonucleotide that is attached to a polynucleotide and is used to identify and/or track the polynucleotide in a reaction. Usually, an oligonucleotide tag is attached to the 3′- or 5′-end of a polynucleotide to form a linear conjugate, sometime referred to herein as a “tagged polynucleotide,” or equivalently, an “oligonucleotide tag-polynucleotide conjugate,” or “tag-polynucleotide conjugate.” Oligonucleotide tags may vary widely in size and compositions; the following references provide guidance for selecting sets of oligonucleotide tags appropriate for particular embodiments: Brenner, U.S. Pat. No. 5,635,400; Brenner et al., Proc. Natl. Acad. Sci., 97: 1665; Shoemaker et al. (1996) Nature Genetics, 14:450; Morris et al., EP Patent Pub. 0799897A1; Wallace, U.S. Pat. No. 5,981,179; and the like.

In one embodiment, an amplifiable probe of the invention comprises at least one oligonucleotide tag that is replicated and labeled to produce a labeled oligonucleotide probe. In certain exemplary embodiments, the label is a retrievable label. The nature of the label on an oligonucleotide tag can be based on a wide variety of physical or chemical properties including, but not limited to, light absorption, fluorescence, chemiluminescence, electrochemiluminescence, mass, charge, and the like. The signals based on such properties can be generated directly or indirectly. For example, a label can be a fluorescent molecule covalently attached to an amplified oligonucleotide tag that directly generates an optical signal. Alternatively, a label can comprise multiple components, such as a hapten-antibody complex, that, in turn, may include fluorescent dyes that generated optical signals, enzymes that generate products that produce optical signals, or the like. In certain aspects, the label on an oligonucleotide tag is a fluorescent label that is directly or indirectly attached to an amplified oligonucleotide tag.

Fluorescent labels and their attachment to oligonucleotides, such as oligonucleotide tags, are described in many reviews, including Haugland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); Wetmur, Critical Reviews in Biochemistry and Molecular Biology, 26:227-259 (1991); and the like. Particular methodologies applicable to the invention are disclosed in the following sample of references: Fung et al., U.S. Pat. No. 4,757,141; Hobbs, Jr., et al. U.S. Pat. No. 5,151,507; Cruickshank, U.S. Pat. No. 5,091,519. In one aspect, one or more fluorescent dyes are used as labels for labeled target sequences, e.g., as disclosed by Menchen et al., U.S. Pat. No. 5,188,934 (4,7-dichlorofluorscein dyes); Begot et al., U.S. Pat. No. 5,366,860 (spectrally resolvable rhodamine dyes); Lee et al., U.S. Pat. No. 5,847,162 (4,7-dichlororhodamine dyes); Khanna et al., U.S. Pat. No. 4,318,846 (ether-substituted fluorescein dyes); Lee et al., U.S. Pat. No. 5,800,996 (energy transfer dyes); Lee et al., U.S. Pat. No. 5,066,580 (xanthine dyes): Mathies et al., U.S. Pat. No. 5,688,648 (energy transfer dyes); and the like. Labelling can also be carried out with quantum dots, as disclosed in the following patents and patent publications: U.S. Pat. Nos. 6,322,901; 6,576,291; 6,423,551; 6,251,303; 6,319,426; 6,426,513; 6,444,143; 5,990,479; 6,207,392; 2002/0045045; 2003/0017264; and the like. As used herein, the term “fluorescent label” includes a signaling moiety that conveys information through the fluorescent absorption and/or emission properties of one or more molecules. Such fluorescent properties include fluorescence intensity, fluorescence life time, emission spectrum characteristics, energy transfer, and the like.

Commercially available fluorescent nucleotide analogues readily incorporated into the labeling oligonucleotides include, for example, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHODAMINE GREEN™-5-dUTP, OREGON GREENR™ 488-5-dUTP, TEXAS RED™-12-dUTP, BODIPY TM 630/650-14-dUTP, BODIPY TM 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY TM FL-14-UTP, BODIPY TMR-14-UTP, BODIPY TM TR-14-UTP, RHODAMINE GREEN™-5-UTP, ALEXA FLUOR™ 488-5-UTP, LEXA FLUOR™ 546-14-UTP (Molecular Probes, Inc. Eugene, Oreg.). Protocols are available for custom synthesis of nucleotides having other fluorophores. Henegariu et al., “Custom Fluorescent-Nucleotide Synthesis as an Alternative Method for Nucleic Acid Labeling,” Nature Biotechnol. 18:345-348 (2000).

Other fluorophores available for post-synthetic attachment include, inter alia, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), and Cy2, Cy3.5, Cy5.5, and Cy7 (Amersham Biosciences, Piscataway, N.J. USA, and others).

FRET tandem fluorophores may also be used, such as PerCP-Cy5.5, PE-Cy5, PE-Cy5.5, PE-Cy7, PE-Texas Red, and APC-Cy7; also, PE-Alexa dyes (610, 647, 680) and APC-Alexa dyes.

Metallic silver particles may be coated onto the surface of the array to enhance signal from fluorescently labeled oligos bound to the array. Lakowicz et al. (2003) Bio Techniques 34:62.

Biotin, or a derivative thereof, may also be used as a label on a detection oligonucleotide, and subsequently bound by a detectably labeled avidin/streptavidin derivative (e.g. phycoerythrin-conjugated streptavidin), or a detectably labeled anti-biotin antibody. Digoxigenin may be incorporated as a label and subsequently bound by a detectably labeled anti-digoxigenin antibody (e.g. fluoresceinated anti-digoxigenin). An aminoallyl-dUTP residue may be incorporated into a detection oligonucleotide and subsequently coupled to an N-hydroxy succinimide (NHS) derivatized fluorescent dye, such as those listed supra. In general, any member of a conjugate pair may be incorporated into a detection oligonucleotide provided that a detectably labeled conjugate partner can be bound to permit detection. As used herein, the term antibody refers to an antibody molecule of any class, or any sub-fragment thereof, such as an Fab.

Other suitable labels for detection oligonucleotides may include fluorescein (FAM), digoxigenin, dinitrophenol (DNP), dansyl, biotin, bromodeoxyuridine (BrdU), hexahistidine (6×His), phosphor-amino acids (e.g. P-tyr, P-ser, P-thr), or any other suitable label. In one embodiment the following hapten/antibody pairs are used for detection, in which each of the antibodies is derivatized with a detectable label: biotin/α-biotin, digoxigenin/a-digoxigenin, dinitrophenol (DNP)/α-DNP, 5-Carboxyfluorescein (FAM)/α-FAM.

As mentioned above, oligonucleotide tags can be indirectly labeled, especially with a hapten that is then bound by a capture agent, e.g., as disclosed in Holtke et al., U.S. Pat. Nos. 5,344,757; 5,702,888; and 5,354,657; Huber et al., U.S. Pat. No. 5,198,537; Miyoshi, U.S. Pat. No. 4,849,336; Misiura and Gait, PCT publication WO 91/17160; and the like. Many different hapten-capture agent pairs are available for use with the invention, either with a target sequence or with a detection oligonucleotide used with a target sequence, as described below. Exemplary, haptens include, biotin, des-biotin and other derivatives, dinitrophenol, dansyl, fluorescein, CY5, and other dyes, digoxigenin, and the like. For biotin, a capture agent may be avidin, streptavidin, or antibodies. Antibodies may be used as capture agents for the other haptens (many dye-antibody pairs being commercially available, e.g., Molecular Probes, Eugene, Oreg.).

“Polymorphism” or “genetic variant” means a substitution, inversion, insertion, or deletion of one or more nucleotides at a genetic locus, or a translocation of DNA from one genetic locus to another genetic locus. In one aspect, polymorphism means one of multiple alternative nucleotide sequences that may be present at a genetic locus of an individual and that may comprise a nucleotide substitution, insertion, or deletion with respect to other sequences at the same locus in the same individual, or other individuals within a population. An individual may be homozygous or heterozygous at a genetic locus; that is, an individual may have the same nucleotide sequence in both alleles, or have a different nucleotide sequence in each allele, respectively. In one aspect, insertions or deletions at a genetic locus comprises the addition or the absence of from 1 to 10 nucleotides at such locus, in comparison with the same locus in another individual of a population (or another allele in the same individual). Usually, insertions or deletions are with respect to a major allele at a locus within a population, e.g., an allele present in a population at a frequency of fifty percent or greater.

“Primer” includes an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Usually primers are extended by a DNA polymerase. Primers usually have a length in the range of between 3 to 36 nucleotides, also 5 to 24 nucleotides, also from 14 to 36 nucleotides. Primers within the scope of the invention can be universal primers or non-universal primers. Pairs of primers can flank a sequence of interest or a set of sequences of interest. Primers and probes can be degenerate in sequence. Primers within the scope of the present invention bind adjacent to the target sequence, whether it is the sequence to be captured for analysis, or a tag that it to be copied.

“Solid support,” “support,” and “solid phase support” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. Microarrays usually comprise at least one planar solid phase support, such as a glass microscope slide. Semisolid supports and gel supports are also useful in the present invention, especially when polony amplification is used.

“Specific” or “specificity” in reference to the binding of one molecule to another molecule, such as a target sequence to a probe, means the recognition, contact, and formation of a stable complex between the two molecules, together with substantially less recognition, contact, or complex formation of that molecule with other molecules. In one aspect, “specific” in reference to the binding of a first molecule to a second molecule means that to the extent the first molecule recognizes and forms a complex with another molecules in a reaction or sample, it forms the largest number of the complexes with the second molecule. Preferably, this largest number is at least fifty percent. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like. As used herein, “contact” in reference to specificity or specific binding means two molecules are close enough that weak non-covalent chemical interactions, such as van der Waal forces, hydrogen bonding, base-stacking interactions, ionic and hydrophobic interactions, and the like, dominate the interaction of the molecules.

“Spectrally resolvable” in reference to a plurality of fluorescent labels means that the fluorescent emission bands of the labels are sufficiently distinct, i.e., sufficiently non-overlapping, that molecular tags to which the respective labels are attached can be distinguished on the basis of the fluorescent signal generated by the respective labels by standard photodetection systems, e.g., employing a system of band pass filters and photomultiplier tubes, or the like, as exemplified by the systems described in U.S. Pat. Nos. 4,230,558; 4,811,218, or the like, or in Wheeless et al., pgs. 21-76, in Flow Cytometry: Instrumentation and Data Analysis (Academic Press, New York, 1985). In one aspect, spectrally resolvable organic dyes, such as fluorescein, rhodamine, and the like, means that wavelength emission maxima are spaced at least 20 nm apart, and in another aspect, at least 40 nm apart. In another aspect, chelated lanthanide compounds, quantum dots, and the like, spectrally resolvable means that wavelength emission maxima are spaced at least 10 nm apart, and in a further aspect, at least 15 nm apart.

“Tₘ” is used in reference to “melting temperature.” Melting temperature is the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation. Tₘ=81.5+0.41 (% G+C), when a nucleic acid is in aqueous solution at 1 M NaCl (see e.g., Anderson and Young, “Quantitative Filter Hybridization,” in Nucleic Acid Hybridization (1985). Other references (e.g., Allawi, H. T. & Santa Lucia, J., Jr., Biochemistry 36, 10581-94 (1997)) include alternative methods of computation which take structural and environmental, as well as sequence characteristics into account for the calculation of Tₘ.

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention. The contents of all references, patents and published patent applications cited throughout this application are hereby incorporated by reference in their entirety for all purposes.

The following examples are set forth as being representative of the present invention. These examples are not to be construed as limiting the scope of the invention as these and other equivalent embodiments will be apparent in view of the present disclosure, figures and accompanying claims.

### Example I

### Enrichment of Target Nucleic Acid Sequences

A single microarray will be designed and synthesized with oligonucleotide sequences (e.g., average 70 nucleotides in length) that specifically target certain regions in a genome. For example, when exon-containing regions are targeted, each of the oligonucleotide sequences will comprise an exon-specific sequence flanked by two common sequences (e.g., 15-mers). The length of exon-specific sequences will be approximately 40-mer, and will be adjusted for isothermal hybridization. A 2.1 million-feature array would allow an exonome to be tiled at an average of 1.4× coverage (60 million basepairs of exons/40 basepairs per probe=1.5 million probes; 2.1 million/1.5 million=1.4). Each exon, on average, will be tiled with eight probes for capturing. More than one microarray can be synthesized if deeper coverage of tiling is desired.

To circumvent the concern of low hybridization efficiency on a microarray surface and to make the approach more cost effective, probes synthesized on the microarray will be amplified. Two steps will be utilized to accomplish this. First is to use one of the common sequences as the primer to synthesize the second strand of the oligonucleotide sequences on the microarray. Next, an additional step will be used to release the second strand DNA from the microarray by denaturing, followed by pooling all probes in a single tube. Then, amplification (e.g., PCR) will be performed to amplify the probe sequences with common flanking sequences as primers. One of the primers will be labeled (e.g., 5′ biotinylated) and phosphorothioate protected, and the other will be 5′ phosphorylated. The PCR product will then be treated with λ exonuclease to digest the 5′ phosphorylated DNA strand.

The 5′ biotinylated strand will remain intact and will be hybridized with sheared and end-polished genomic DNA with adapters ligated on both ends. The hybridization mixture will be added with streptavidin coated Dynal magnetic beads (Invitrogen, CA). Only the biotinylated probes with hybridized genomic fragments will be bound to the beads. The genomic fragments will be released from the beads by denaturing, and amplified with common adapter primers. The amplified DNA can then be subjected to sequencing directly or be concatenated, sheared and adapter ligated prior to sequencing.

### Example II

### RCA Generated ssDNA Assisted Capturing Strategy

In contrast to other solution phase enrichment methods (Porreca et al, 2007), this strategy eliminates the necessity of a post oligonucleotide processing step for the generation of ssDNA and generates large amount of hybridization probes for the use in the enrichment step (FIG. 3). RCA generated ssDNA will be directly used for enriching target DNA. Captured DNA will be directly subjected to DNA sequencing.

1) 70-mer oligonucleotide sequences will be synthesized on a microarray. Target DNA sequences (e.g., 40-mer oligonucleotide sequences) will be flanked by common sequences (e.g., 15-mer oligonucleotide sequences). The second strand of the oligonucleotide sequences will be copied by extending one primer.
2) The synthesized second strands will be collected by denaturing, and will serve as templates for RCA amplification. The collected DNA will be 5′ phosphorylated, circularized by circ-ligase, and then subject to the RCA reaction. An alternative method will be to use a “guide” oligonucleotide sequence to bring two ends of an oligonucleotide together with Amp ligase.
3) During the RCA reaction, biotinylated primer and/or biotinylated dNTP (NEB or Invitrogen) will optionally be added to provide a retrievable label to purify captured DNA molecules. The biotinylated ssDNA will be hybridized with target DNA (e.g., genomic DNA that is randomly sheared, end repaired, and ligated with adapters). After hybridization, streptavidin coated magnetic beads will be added to enrich selected genomic DNA.
3′) During the RCA reaction (without adding biotinylated primer or biotinylated dNTP), RCA products will be saturated up to the point of precipitation by either performing hyper-branched RCA, or long period linear RCA. The precipitated RCA products will be used to enrich one or more target DNA and/or RNA regions. Centrifugation at a high speed will be performed to separate unbound DNA molecules in solution phase and target DNA bound with the RCA processed probe molecules. The RCA produced ssDNA will be hybridized with genomic DNA (e.g., DNA that is randomly sheared, end repaired, and ligated with adapters).
(4) The hybridized genomic DNA will be amplified using adapter sequences and subjected to sequencing.

What is claimed is:

1. A method for enriching a target nucleic acid sequence comprising the steps of:
providing a double stranded oligonucleotide probe having a primer sequence at each 5′ end, wherein one strand includes a region that is complementary to the target nucleic acid sequence and includes a primer sequence having retrievable label and a phosphorothioate cap at its 5′ end, and wherein the primer sequence at the 5′ end of the other strand is phosphorylated;
amplifying the probe;
cleaving the strand having the 5′ phosphorylation to generate a single stranded probe having a 5′ retrievable label;
hybridizing the single stranded probe having a 5′ retrievable label to a target nucleic acid sequence of interest;
enriching the target nucleic acid sequence of interest by binding the 5′ retrievable label of the hybridized, single stranded probe having a 5′ retrievable label to a substrate; and
releasing the nucleic acid sequence of interest from the substrate by denaturing.

2. The method of claim 1, wherein the step of amplifying is performed by PCR.

3. The method of claim 1, wherein the retrievable label is biotin.

4. The method of claim 3, wherein step of enriching is performed by binding the biotin to streptavidin attached to a substrate.

5. The method of claim 4, wherein the substrate is a magnetic bead.

6. The method of claim 1, wherein λ exonuclease is used to cleave the strand having the 5′ phosphorylation.

7. The method of claim 1, wherein the target nucleic acid sequence is genomic DNA or genomic RNA.

8. A method for enriching a target nucleic acid sequence comprising the steps of:
providing a circularized probe having a central region that is complementary to the target nucleic acid sequence;
amplifying the probe by rolling circle amplification in the presence of a retrievable label to generate single stranded, amplified probe having a retrievable label attached thereto;
hybridizing the single stranded, amplified probe having a retrievable label attached thereto to a target nucleic acid sequence of interest;
enriching the target nucleic acid sequence of interest by binding the retrievable label to a substrate; and
releasing the nucleic acid sequence of interest from the substrate by denaturing.

9. The method of claim 8, wherein the retrievable label is added to the probe using a labeled primer or a labeled dNTP.

10. The method of claim 8, wherein the retrievable label is biotin.

11. The method of claim 10, wherein the step of enriching is performed by binding the biotin label to streptavidin bound to a substrate.

12. The method of claim 11, wherein the substrate is a magnetic bead.

13. The method of claim 8, wherein the target nucleic acid sequence is genomic DNA or genomic RNA.

14. A method for enriching a target nucleic acid sequence comprising the steps of:
providing a double stranded oligonucleotide probe having a primer sequence at each 5′ end, wherein one strand includes a region that is complementary to the target nucleic acid sequence and includes a primer sequence having retrievable label, and wherein the primer sequence at the 5′ end of the other strand is phosphorylated;
amplifying the probe using an asymmetric ratio of forward primer to reverse primer to generate a single stranded probe having a 5′ retrievable label;
hybridizing the single stranded probe having a 5′ retrievable label to a target nucleic acid sequence of interest;
enriching the target nucleic acid sequence of interest by binding the 5′ retrievable label of the hybridized, single stranded probe having a 5′ retrievable label to a substrate; and
releasing the nucleic acid sequence of interest from the substrate by denaturing.

15. The method of claim 14, wherein the step of amplifying is performed by PCR.

16. The method of claim 14, wherein the single stranded probe is single stranded DNA.

17. The method of claim 14, wherein the ratio of forward primer to reverse primer is between 100:1 and 2:1.

18. The method of claim 14, wherein the ratio of forward primer to reverse primer is between 20:1 and 2:1.

19. The method of claim 14, wherein the ratio of forward primer to reverse primer is 10:1.

20. The method of claim 14, wherein the target nucleic acid sequence is genomic DNA or genomic RNA.
